(19) United States
(12) Patent Application Publication
(10) Pub. No.: US 2025/0331766 A1
(43) Pub. Date: Oct. 30, 2025

(54) METHOD FOR PROVIDING DYSKINESIA-RELATED INFORMATION AND DEVICE USED THEREFOR

(71) Applicants: SEOUL NATIONAL UNIVERSITY HOSPITAL, Seoul (KR); KOREA UNIVERSITY RESEARCH AND BUSINESS FOUNDATION, SEJONG CAMPUS, Sejong (KR)

(72) Inventors: Won-Seok KIM, Seongnam-si, Gyeonggi-do (KR); Nam-Jong PAIK, Seongnam-si, Gyeonggi-do (KR); Han-Jeong HWANG, Sejong (KR); Miseon SHIM, Sejong (KR)

(21) Appl. No.: 18/728,903

(22) PCT Filed: Sep. 13, 2022
(86) PCT No.: PCT/KR2022/013629

§ 371 Date: Sep. 18, 2024

(30) Foreign Application Priority Data
Jan. 19, 2022 (KR) 10-2022-0007841

(51) Int. Cl. A61B 5/00; A61B 5/291; A61N 1/36; G16H 50/20

(52) U.S. Cl. CPC A61B 5/4082; A61B 5/291; A61N 1/36003; A61N 1/36067; G16H 50/20

(57) ABSTRACT

One aspect of the present disclosure relates to a method for providing motor impairment-related information, comprising the steps of: (a) measuring a brain network index according to movement performance of an affected side part of a subject; and (b) comparing the brain network index measured in step (a) with a brain network index according to movement performance of an unaffected side part of the subject. In addition, another aspect of the present disclosure relates to a device for providing a motor impairment-related information that provides a brain network index obtained according to movement performance of a subject. The method or device for providing motor impairment-related information of the present invention according to the present disclosure has an excellent effect of providing information on prognosis for motor impairment, motor impairment-related diagnosis, or motor impairment-related rehabilitation.

### FIELD OF THE INVENTION

The present invention discloses a method for providing motor impairment-related information and devices used therefor.

[National research and development project that supported this invention]

[Assignment serial number] 1711135187

[Assignment number] 2020R1A4A1017775

[Ministry name] Ministry of Science and ICT

[Name of project management (professional) organization] National Research Foundation of Korea

[Research project name] Group research support (R&D)

[Research project title] Artificial intelligence-based user-responsive interaction laboratory

[Contribution rate] 1/1

[Name of project carrying out organization] Kumoh Institute of Technology

[Research period] 2021.06.01 ˜ 2022.02.28

### BACKGROUND OF THE INVENTION

Upper limb impairment is one of the most common impairments after stroke. This upper limb impairment is associated with abnormal neural activity revealed by electroencephalography (EEG)-based studies. Abnormal power spectral density (PSD) in the alpha (8-12 Hz) and low-beta (12-20 Hz) frequency bands when moving the affected body part is typically seen in stroke patients with upper limb impairment.

Previous studies have shown that stroke patients showed weaker alpha and low-beta event-related desynchronization (ERD) patterns in primary sensorimotor and supplementary motor areas when moving the impaired upper limb than healthy controls. Additionally, stroke patients showed a stronger ERD pattern when moving the affected body part in the undamaged contralesional hemisphere than in the ipsilesional hemisphere. These altered neural activities of stroke patients were explained by task-specific EEG power spectrum-based features. However, due to the complex communication between different brain regions, fragmentary PSDs obtained from individual brain regions were not sufficient to fully explain the abnormal brain mechanisms in stroke patients.

Quantifying functional connectivity between brain regions can provide more in-depth information to understand brain mechanisms when moving body parts with motor impairment in stroke patients. According to previous studies, stroke patients showed weaker whole-brain interactions in the beta frequency band than healthy controls. In another study, stroke patients showed higher strength of directional functional connectivity flow from the contralesional hemisphere to the ipsilesional hemisphere when moving their impaired hand, whereas healthy controls showed the opposite trend. These results indicate that the contralesional hemisphere of stroke patients is more involved in the organization of hand motors with motor impairment than the ipsilesional hemisphere. However, these conventional EEG-based connectivity studies only focused on connectivity between brain regions and were not sufficient to investigate information processing in brain network structures.

Some conventional studies used binary network analysis using resting-state EEG to analyze altered brain network properties in stroke patients compared to healthy controls. However, because these previous studies focused on the resting state rather than the condition of moving the motor-impaired body part, there is a limitation in that they cannot analyze how information is processed between the ipsilesional and contralesional hemispheres during motor performance. More importantly, these prior studies used binary network analysis, which converts the original functional connectivity patterns, which have continuous values between 0 and 1, into values of 0 or 1. This previous study lost important information because it performed transformations using arbitrary thresholds without a standard way to determine which interactions were less beneficial than others.

Considering this background, the present inventors completed the present invention by using weighted network analysis to study abnormal brain networks during the performance of hand movements with motor impairment compared to hand movements without motor impairment.

### DETAILED DESCRIPTION OF THE INVENTION

### Technical Challenges

In one aspect, the present invention seeks to provide a method of providing motor impairment-related information.

In another aspect, the present invention seeks to provide a device for providing motor impairment-related information.

In another aspect, the present invention seeks to provide a computer-readable recording medium that performs the method for providing motor impairment-related information.

### Technical Solution

In one aspect, the present invention provides a method for providing motor impairment-related information, comprising: (a) a step of measuring a brain network index according to movement performance of an affected side part of a subject: and (b) a step of comparing the brain network index measured in step (a) with a brain network index according to movement performance of an unaffected side part of the subject.

In one aspect, the present invention provides a method for providing motor impairment-related information, comprising: (a) a step of measuring a brain network index according to movement performance of a subject with motor impairment: and (b) a step of comparing the brain network index measured in step (a) with a brain network index according to movement performance of a normal subject.

In an exemplary embodiment, the brain network index may comprise one or more selected from the group consisting of strength, clustering coefficient, path length, and small-worldness.

In an exemplary embodiment, the subject may be a subject performing a preset task in step (a).

In an exemplary embodiment, the brain network index may be measured in one or more of an alpha wave region and a low-beta wave region.

In an exemplary embodiment, the step (a) may be performed by electroencephalography (EEG).

In an exemplary embodiment, the motor impairment may be a stroke.

In an exemplary embodiment, the subject in step (a) may have upper limb paralysis following a stroke.

In an exemplary embodiment, the motor impairment-related information may comprise information regarding one or more of prognosis prediction for the motor impairment, motor impairment-related diagnosis, and motor impairment-related rehabilitation.

In one aspect, the present invention provides a device for providing motor impairment-related information that provides a brain network index obtained according to movement performance of a subject.

In an exemplary embodiment, the brain network index may be expressed as (i) a ratio of a brain network index according to movement performance of the affected side of the subject to a brain network index according to movement performance of the unaffected side part of the subject, or (ii) a ratio of a brain network index according to movement performance of a subject with motor impairment to a brain network index according to movement performance of a normal subject.

In one aspect, the present invention provides a device comprising: the device for providing motor impairment-related information: and one or more of a brain stimulation unit that is connected to the device for providing motor impairment-related information and performs brain stimulation based on the brain network index and a rehabilitation assistant unit that assists rehabilitation of motor impairment.

In an exemplary embodiment, the brain stimulation unit may perform one or more of brain electrical stimulation, brain magnetic stimulation, and neurofeedback, and the rehabilitation assistant unit may comprise a robot arm that assists rehabilitation.

In an exemplary embodiment, the device may further comprise an instruction device comprising an AR or VR device for instructing a subject movement task.

In one aspect, the present invention provides a computer-readable recording medium that is readable by a computer and stores program instructions operable by the computer, wherein when the program instructions are executed by a processor of the computer, the computer-readable recording medium causes the processor to perform the method for providing motor impairment-related information.

### Effects of the Invention

In one aspect, the method or device for providing motor impairment-related information according to an embodiment of the present invention is excellent in providing information on prognosis prediction for the motor impairment, motor impairment-related diagnosis, or motor impairment-related rehabilitation, etc.

In one aspect, the method or device for providing motor impairment-related information according to an embodiment of the present invention can be used clinically to determine motor impairment-related rehabilitation by predicting the prognosis for motor impairment, etc. and selecting an appropriate treatment method.

### BRIEF DESCRIPTION OF THE DRAWINGS

FIG. 1A is a schematic diagram of a hand movement task performed by a subject according to an example of the present invention.

FIG. 1B is a schematic diagram showing the location of electrodes used in one example of the present invention. In FIG. 1B, the blue box represents the left ROI and the green box represents the right ROI, and ROI means regions of interest.

FIG. 2 shows the average topography and quantified low-beta ERD for a subject when performing motor impairment-and non-motor impairment-hand movements tasks in one example of the present invention (CON refers to the undamaged contralesional hemisphere and IPSI refers to the damaged ipsilesional hemisphere).

FIG. 3A to 3H are graphs comparing the network indices between the damaged ipsilesional hemisphere and the undamaged contralesional hemisphere in one example of the present invention.

FIG. 4A to 4H are graphs showing the correlation between functional network indices and FMA score in one example of the present invention.

### MODE FOR CARRYING OUT THE INVENTION

Hereinafter, the present invention will be described in detail.

In this specification, “motor impairment-related information” means information about one or more of prognosis prediction for the motor impairment. motor impairment-related diagnosis, and motor impairment-related rehabilitation.

In one aspect, the present invention provides a method for providing motor impairment-related information, comprising: a step of measuring a brain network index according to movement performance of an affected side part of a subject: and a step of comparing the brain network index measured in step (a) with a brain network index according to movement performance of an unaffected side part of the subject.

In one aspect, the present invention provides a method for providing motor impairment-related information, comprising: a step of measuring a brain network index according to movement performance of a subject with motor impairment: and a step of comparing the brain network index measured in step (a) with a brain network index according to movement performance of a normal subject. The normal subject refers to a subject who does not have a motor impairment.

In one embodiment, the brain network index may comprise one or more selected from the group consisting of strength, clustering coefficient, path length, and small-worldness.

In this specification, strength refers to how strongly brain regions are connected to each other, and the clustering coefficient refers to how well one brain region is clustered with neighboring regions. Additionally, path length refers to the overall connectivity of the entire brain network structure, and small-worldness refers to how a brain network operates efficiently when transferring information from one region to another compared to a random network. In one implementation, in step (a), the subject may be performing a preset task in step (a).

In one embodiment, the brain network index may be measured in one or more of an alpha wave region and a low-beta wave region. The alpha wave region may refer to a brain wave region with a frequency of 8 to 12 Hz, and the low-beta wave region may refer to a brain wave region with a frequency of 12 to 20 Hz.

In one embodiment, the step (a) may be performed by electroencephalography (EEG).

In one embodiment, the subject in step (a) may be a subject with upper limb paralysis after a stroke.

In one aspect, the present invention provides a device for providing motor impairment-related information that provides a brain network index obtained according to movement performance of a subject.

In one embodiment, the brain network index may be expressed as (i) a ratio of a brain network index according to movement performance of the affected side of the subject to a brain network index according to movement performance of the unaffected side part of the subject, or (ii) a ratio of a brain network index according to movement performance of a subject with motor impairment to a brain network index according to movement performance of a normal subject.

In one aspect, the present invention provides a device comprising a motor impairment-related information providing device that provides a brain network index obtained according to movement performance of a subject: and one or more of a brain stimulation unit that is connected to the motor impairment-related information providing device and performs brain stimulation based on the brain network index and a rehabilitation assistant unit that assists rehabilitation of motor impairment.

In one embodiment, the brain stimulation unit may perform one or more of brain electrical stimulation, brain magnetic stimulation, and neurofeedback.

In one embodiment, the rehabilitation assistant unit may comprise a robot arm that assists rehabilitation.

In one implementation, the device may further comprise an instruction device comprising an Augmented Reality (AR) or Virtual Reality (VR) device for instructing a subject movement task.

In one aspect, the present invention provides a computer-readable recording medium causes the processor to perform the method for providing motor impairment-related information according to any one of claims 1 to 9 as the computer-readable recording medium that is readable by a computer and stores program instructions operable by the computer, wherein when the program instructions are executed by a processor of the computer.

The computer may be a computing device, such as a desktop computer, laptop computer, notebook, smart phone, or the like, or any device that may be integrated. A computer is a device that has one or more alternative, special-purpose processors, memory, storage, and networking components (either wireless or wired). The computer may run an operating system such as, for example, Microsoft's Windows-compatible operating system, Apple's OS or iOS, Linux distribution, or Google's Android OS.

The computer-readable recording medium may comprise all types of recording and identification devices that store data that can be read by a computer. Examples of computer-readable recording media include ROM, RAM, CD-ROM, magnetic tape, floppy disk, and optical data storage identification devices, etc. Additionally, the computer-readable recording media may be distributed across computer systems connected to a network, and computer-readable codes may be stored and executed in a distributed manner. Additionally, functional programs, codes, and code segments for implementing this embodiment can be easily understood by those skilled in the art to which this embodiment belongs.

Hereinafter, the configuration and effects of the present invention will be described in more detail through examples. However, the examples below are provided only for illustrative purposes to aid understanding of the present invention, and the category and scope of the present invention are not limited thereto.

### [Example 1] Selection of Subjects

Thirty-four chronic stroke patients were enrolled as subjects in the study according to the present invention. Subjects meet the following criteria: (1) 18-85 years of age: (2) first ischemic or hemorrhagic stroke confirmed by computed tomography (CT) or magnetic resonance imaging (MRI): (3) isolated upper limb weakness: (4) more than 6 months after stroke: and (5) have the ability to provide written consent. All subjects received detailed information about the study, and written consent was obtained from all subjects. This study was approved by the Institutional Review Board of Seoul National University Bundang Hospital and was conducted in accordance with the Code of Ethics of the World Medical Association.

### [Example 2] Diagnosis and Analysis of the Subject'S Brain Network Index

### Clinical Data Collection

To evaluate the motor function of the subjects (stroke patients), the FMA score was obtained, a comprehensive motor impairment index consisting of 5 domains, where the upper limb motor domain (0-66) was used. Subjects' structural images (e.g., CT or MRI) were reviewed by a clinician, and stroke location was classified as cortical, subcortical (radicular, internal capsule, or basal row), or cortical/subcortical.

### Experimental Form

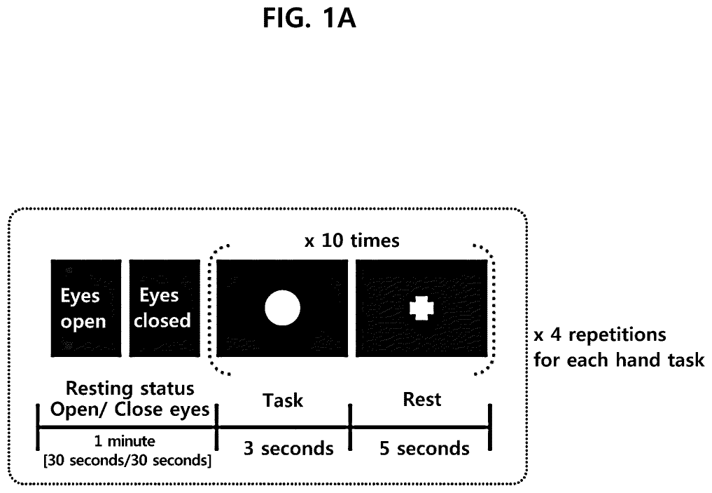

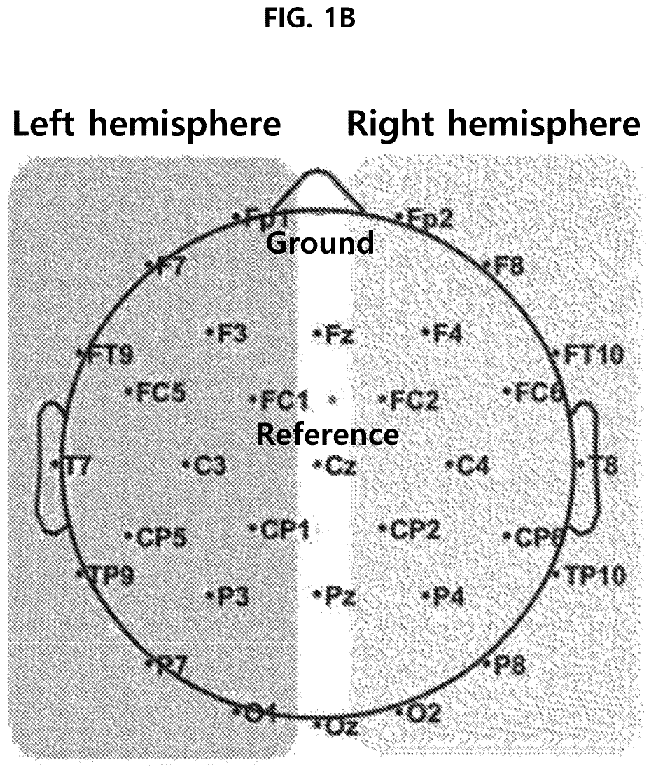

In this study, reaching movements were performed repeatedly, during which EEG associated with the movements was recorded. Subjects sat comfortably in front of the monitor. Before performing the hand movement task, resting EEG was recorded for 1 minute (eyes open—30 seconds, eyes closed—30 seconds). A single trial containing 3 sec of reaching movement and 5 sec of relaxation was repeated 10 times in a single session (FIG. 1A). A total of 8 sessions were performed sequentially, with 4 sessions targeting movements of the hand with motor impairment and the remaining 4 sessions targeting movements of the hand without motor impairment. During the reaching task, patients were asked to look at a fixation cross displayed on the screen to minimize electrooculography (EOG) artifacts.

### EEG Recording and Preprocessing

EEG data were recorded at a sampling rate of 1000 Hz from 32 Ag/AgCl scalp electrodes placed evenly on the scalp. The ground electrode and reference electrode were attached to Fpz and FCz. Because the position of the ground electrode is measured relative to the reference electrode, physiological artifacts such as EOG do not occur when measuring EEG. The location of the reference electrode (FCz) used in this study is relatively insensitive to EOG because it is located around the central region (Cz) of the scalp. However, EEG inherently contains EOG artifacts regardless of reference location. Eye-related artifacts were removed using a mathematical procedure based on principal component analysis (PCA) using the first PCA component of the mean eye movements implemented in Curry 7 (Compumedics, USA) software. EEG data were band-pass filtered from 0.1 to 55 Hz and included a baseline period split from 1 to 3.5 seconds relative to task onset. This was used to correct for inter- and intra-subject EEG variability when calculating event-related spectral perturbations (ERSP). If an epoch exhibited gross artifacts (±100 uV) at one of the electrodes, it was rejected from further analysis. The mean and standard deviation of rejected epochs ranged from 3.69 to 7.15 for the affected hand gesture task and only 1.62 to 3.95 for the unaffected hand gesture task. Only artifact-free EEG data were used for PSD and functional network analysis.

### Individually Optimized Frequency Band Based on IAF

To quantify brain function metrics, we used individually optimized frequency bands defined based on IAF for each of the alpha and low-beta bands. For this purpose. PSD was calculated using the Fast Fourier Transform method using 30 seconds of resting-state eyes-closed EEG data recorded before the movement task. The frequency representing the maximum PSD peak was detected at 7 to 12 Hz at the Oz electrode and was used as IAF. Alpha and low beta frequency bands were defined based on IAF (IAF−2 to IAF+2 Hz for the alpha band and IAF+2 to IAF+11 Hz for the low-beta band). The average alpha and low-beta frequency bands were 7.12-11.12 and 11.12-20.12 Hz, respectively.

### ERSP Analysis

Because alpha and low-beta frequency bands are closely associated with motor tasks, alpha and low-beta event-related desynchronization (ERD) were quantified using the ERSP method for all EEG electrodes. To calculate the spectral power over time, a short-time Fourier transform with a Hanning window size of 250 msec was performed for each trial. The power spectrum of each trial was then normalized with respect to the average power of the baseline period (−1 to 0) second) to examine changes in spectral power values before and after the onset of the reaching movement. The normalized power spectra were then averaged across trials to generate a baseline normalized ERSP map for each subject. Negative values of spectral power were extracted to quantify ERD for two frequency bands: alpha and low-beta bands. Electrodes were grouped into two regions of interest (ROIs) according to hemisphere to examine the effect of brain lesions on ERD amplitudes in motor tasks for impaired and non-impaired hands, respectively (FIG. 1B) (left hemisphere: Fp1, F3: F7, FT9, FC5, FC1, C3, T7, TP9, CP5, CP1, P3, P7, O1: right hemisphere: Fp2, F8, F4, FC2, FC6, FT10, T8, C4, CP2, CP6, TP10, P8, P4 and O2). Midline electrodes were not used in data analysis, such as Fz, Cz, Pz, and Oz. Representative ERD values for each hemisphere were quantified by averaging the corresponding electrode and time for each subject.

### Functional Network Analysis

Functional network analysis was performed to investigate changes in brain activity from a brain network perspective. To calculate a weighted whole-brain network index based on graph theory, a functional connectivity matrix was calculated as a prerequisite. In this study, among various functional connectivity indices, the Hilbert Transform-based PLV (Phase Locking Value) was calculated for the alpha and low-beta bands using own Matlab function. PLV was assessed between all possible pairs of 32 EEG electrodes at each time point during only the task period (0-3.5 seconds) and a PLV matrix was generated by averaging over time. PLV has values between 0 and 1. The higher the PLV, the stronger the connection between two electrodes than other electrode pairs.

In this study, based on graph theory, four weighted network indices were evaluated: (1) strength, (2) clustering coefficient, (3) path length, and (4) small-worldness. The PLV matrix was used in the same way as the graph (G) consisting of a sensor (V) and an edge (E), and PLV represents the weight (w) of the edge between the two sensors. Strength indicates how strongly brain regions are connected to each other, estimated as the sum of the weights of edges connected to the sensor. The strength (local level) for a particular sensor is defined as:

$${S}_{i} = {\sum }_{j \neq i} {w}_{ij}$$

Where i represents a specific sensor and j represents other sensors (j=1, . . . , n−1, n=number of sensors). Global level strength (S) is calculated by averaging strength values from all sensors. Global level strength (S) is calculated by averaging strength values from all sensors. The clustering coefficient indicates how well one brain region is clustered with neighboring regions, and is quantified based on the degree of clustering between the three sensors to create a triangle. To calculate the clustering coefficient for a particular sensor, the sensor's neighboring triangle values must be calculated before:

$${t}_{i} = \frac{1}{2} {\sum }_{j , h ∈ N} ( {w}_{ij} \times {w}_{jh} \times {w}_{hi} \text{?}$$

$$\text{?} \text{indicates text missing or illegible when filed}$$

Where N represents all sensors included in G, and j and h are all possible adjacent sensor pairs forming a triangle with a particular sensor. The local level clustering coefficient for a specific sensor is defined as:

$${C}_{i} = \frac{1}{n} {\sum }_{i ∈ N} \frac{2 {t}_{i}}{{k}_{i} ( {k}_{i} - 1 )}$$

Where n is the number of sensors and kᵢ is the number of all sensors connected to a specific sensor. In this study. since the number of sensors was 32 and each sensor was assumed to be fully connected to other sensors, n and kᵢ were 32 and 31, respectively. The global level clustering coefficient (C) was quantified by averaging the clustering coefficients of all sensors. Path length represents the overall connectivity of the entire network structure (integration). Path length (L) is defined as:

$$L = \frac{1}{n ( n - 1 )} {\sum }_{i , j ∈ N , i \neq j} {d}_{ij}$$

Where dᵢⱼ represents the shortest distance between two sensors (i and j), quantified as the reciprocal of the weight when using a fully connected weight graph.

Small-worldness refers to how a brain network operates cost-efficiently when transferring information from one region to another compared to a random network. To evaluate small-worldness, we normalized both the clustering coefficient and the path length for each index evaluated using a randomly rewired null function connectivity matrix, then calculated a ratio of the normalized clustering coefficient (gamma) to the normalized path length (lambda) to evaluate small-worldness (small-worldness=gamma/lambda). We quantified four network indices in three ROIs: (1) whole brain (both hemispheres), (2) ipsilateral hemisphere only, and (3) cerebral hemisphere only. Additionally, to investigate the specific regional network characteristics of stroke patients during movement, we calculated the local-level network indices of nodule strength and nodule clustering coefficient. All network measurements were calculated using Brain Connectivity Toolbox (BCT: www.brain-connectivity-toolbox.net) based on MATLAB.

### Statistical Analysis

A statistical approach was applied to the ERD and network analysis indices. A two-way repeated measures analysis of variance (rmANOVA) was performed to assess within-subject factors and ERD differences across tasks (hand movement tasks with motor impairment and without motor impairment) and hemispheres (ipsilesional and contralesional hemispheres). For all significant effects, post hoc paired t-test analysis was performed with adjusted two-tailed p-values using the False Discover Rate (FDR) method. Another two-way rmANOVA was performed to examine differences in hemisphere-level network indices with within-subject factors of tasks (hand movement tasks with motor impairment and without motor impairment) and hemisphere (ipsilesional and contralesional hemispheres). Post hoc paired t-test analysis was then performed with two-tailed p-values adjusted using FDR correction. Additionally, we evaluated the relationship between EEG-based functional indices (ERD and network indices) and FMA scores. SPSS version 21.0 (IBM Corp., Armonk, NY, USA) and Matlab were used for statistical analysis.

### ERD Pattern

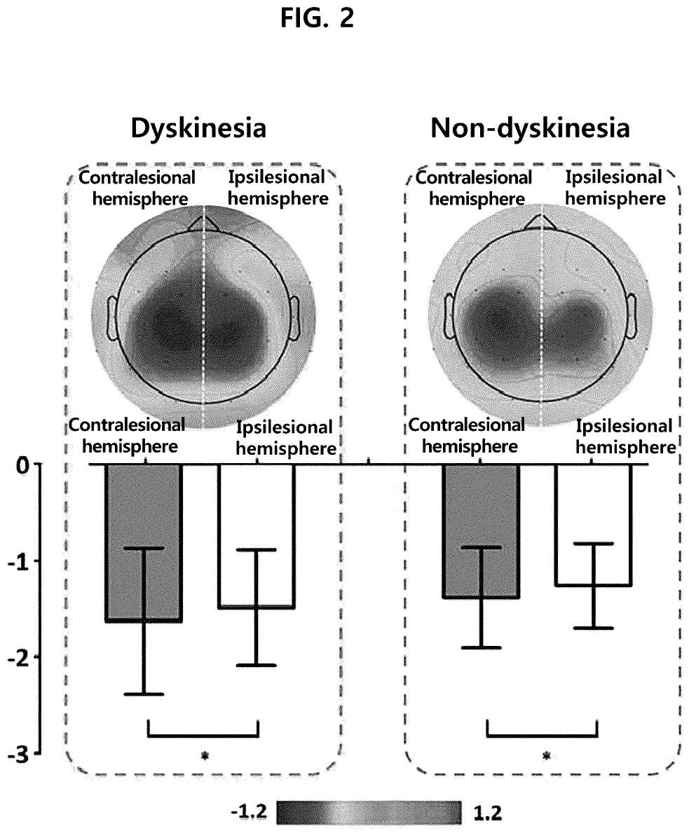

A significant interaction was observed between task and hemisphere in terms of ERD (F=6.396, p=0.016). The interaction was especially noticeable in the low-beta frequency band. Significant ERD differences between hemispheres emerged during hand movements. Topographic low-beta ERD maps for each hand movement task are shown in FIG. 2. A significantly stronger low-beta ERD was observed in the contralesional hemisphere compared to the ipsilesional hemisphere for both the hand motor task with motor impairment (−1.619±0.744 vs. −1.480±0.591, FDR-adjusted p=0.046) and the hand motor task without motor impairment (−1.374+0.513 vs. −1.249±0.434, FDR-adjusted p=0.045).

### Network Characteristics at a Global Level

Significantly altered whole-brain network indices in the low-beta frequency band were found during the hand motor task with motor impairment compared to those found during the hand motor task without motor impairment. It was shown that strength and clustering coefficient were significantly reduced during the motor-impaired hand task compared to the non-impaired hand task. whereas path length was significantly increased during the hand motor task with motor impairment. However, there was no significant difference in small-worldness between the two tasks. These results are shown in Table 1.

TABLE 1

| | Hand motor with | Hand motor without | |
|---|---|---|---|
| | motor impairment | motor impairment | p |
| Strength | 11.196 ± 1.690 | 11.625 ± 1.743 | 0.014 |
| Clustering | 0.342 ± 0.056 | 0.356 ± 0.057 | 0.014 |
| coefficient | | | |
| Path length | 3.249 ± 0.483 | 3.147 ± 0.465 | 0.021 |
| Small-worldness | 0.897 ± 0.032 | 0.894 ± 0.030 | 0.405 |

### Hemisphere-level Network Characteristics

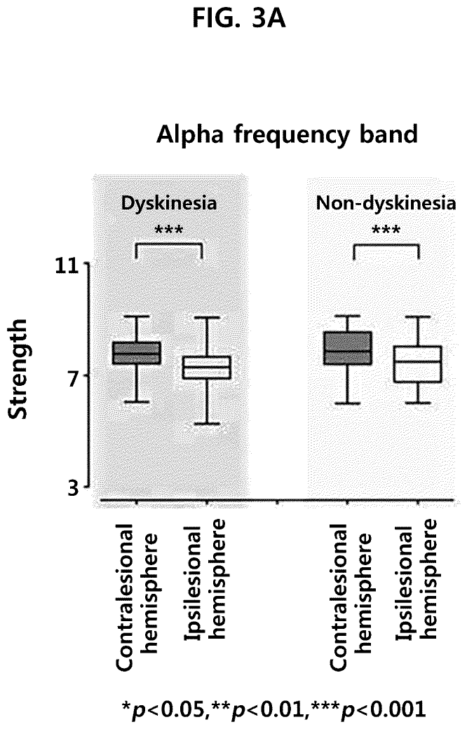

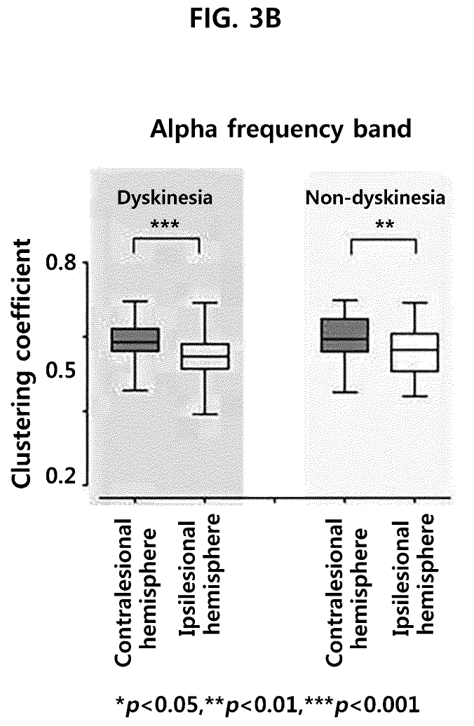

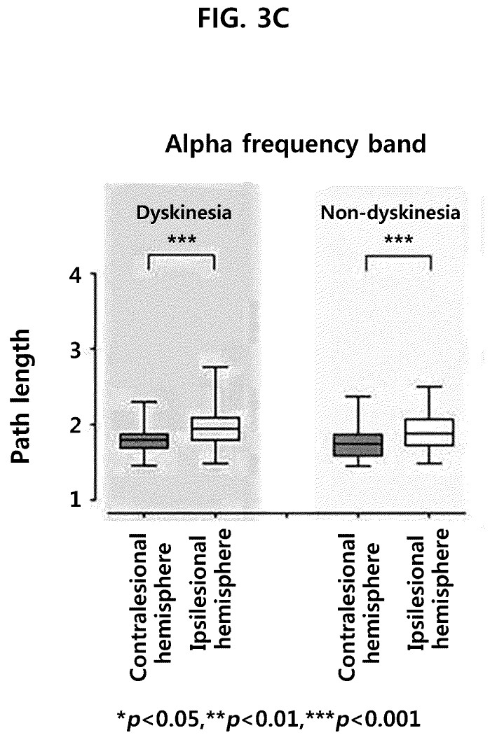

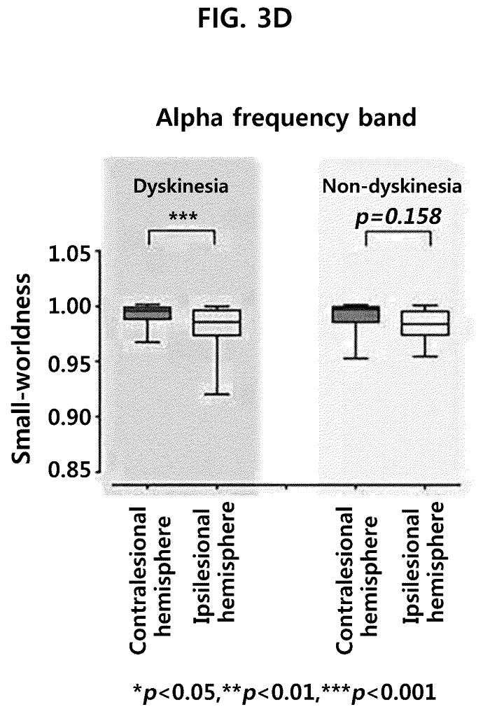

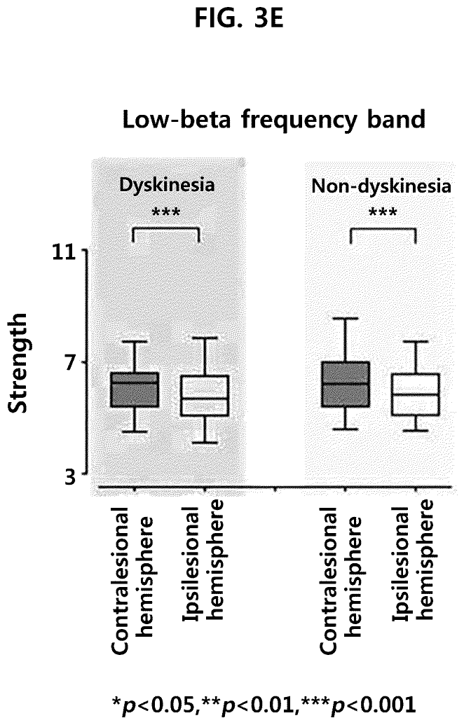

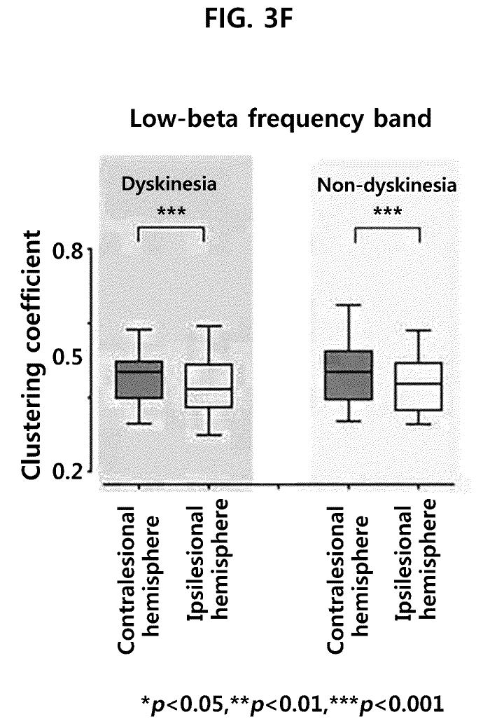

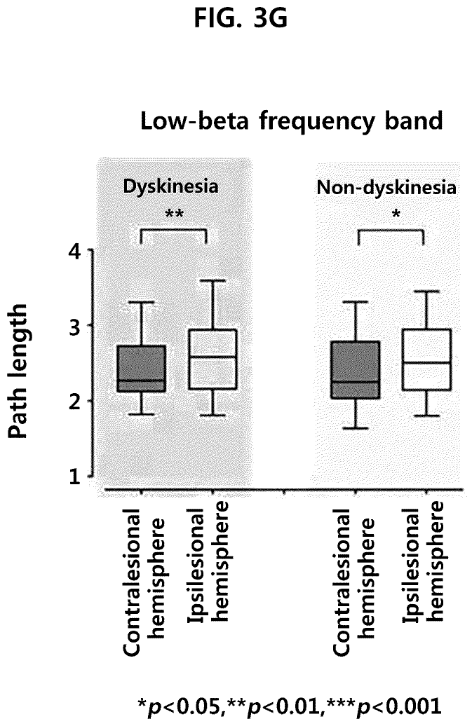

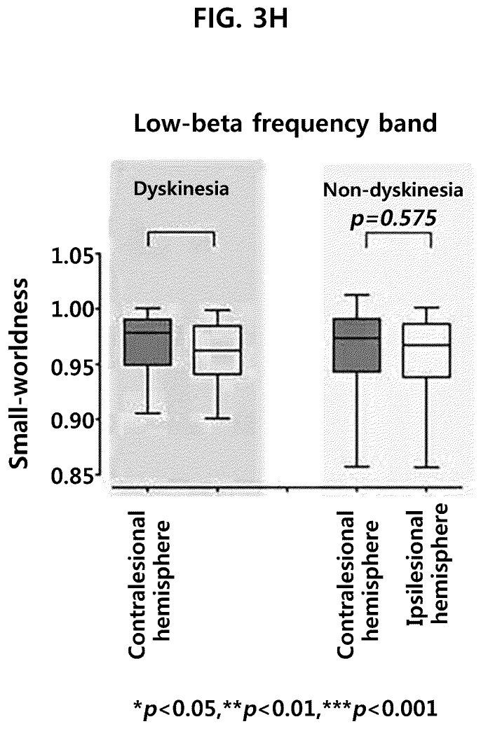

Significant effects of network index were found between tasks and hemispheres in both alpha and low-beta frequency bands (F-statistic, p<0.001 for all comparisons). In both impaired and non-impaired hand movement tasks. the contralesional brain network outperformed the ipsilesional brain network for all three network indices (strength, clustering coefficient, and path length) in both the alpha and low-beta frequency bands (FIGS. 3A to 3H). Strength and clustering coefficient were significantly increased in the contralesional hemisphere compared to the ipsilesional hemisphere, whereas path length was significantly decreased in the contralesional hemisphere. For small-worldness. significant differences were observed in the hand movement task with motor impairment in the alpha and low-beta frequency bands. Small-worldness significantly increased in the contralesional hemisphere compared to the ipsilesional hemisphere.

### Relationship to FMA Score

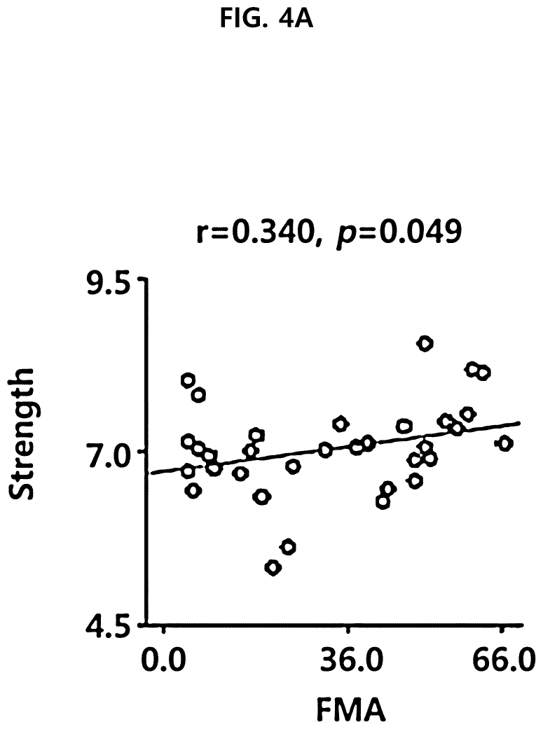

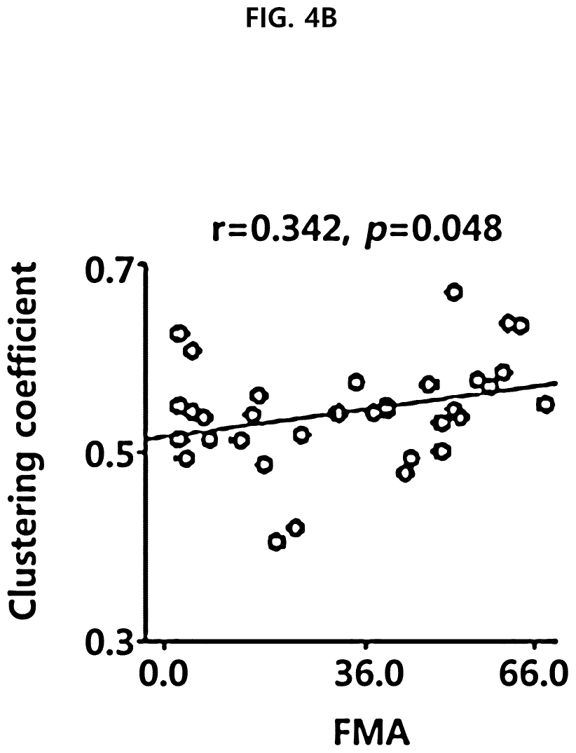

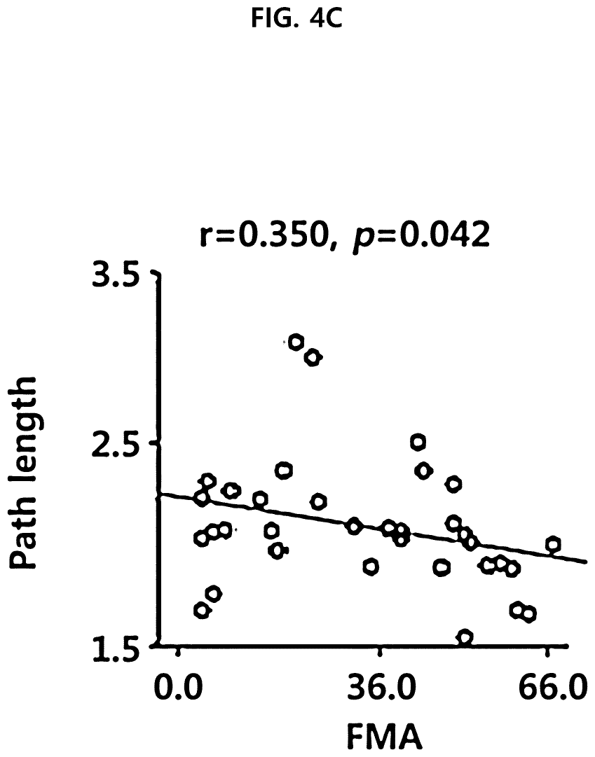

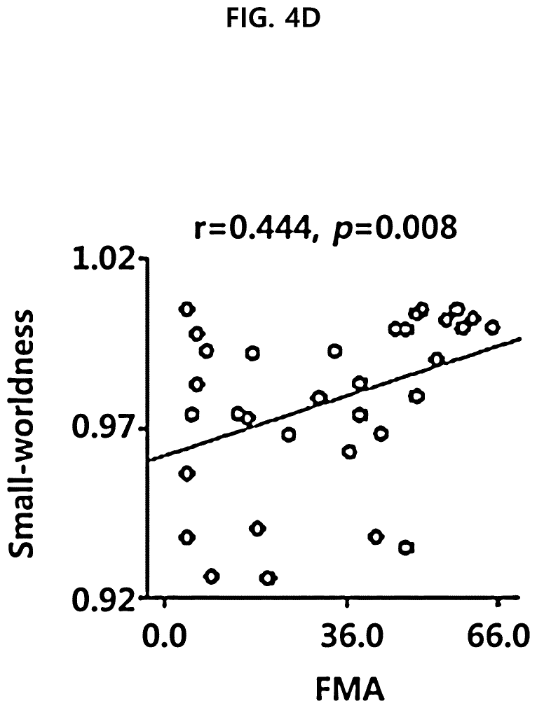

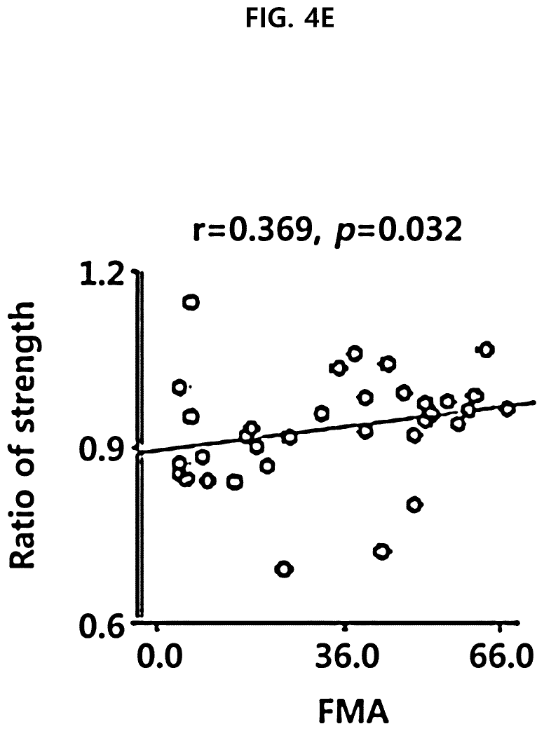

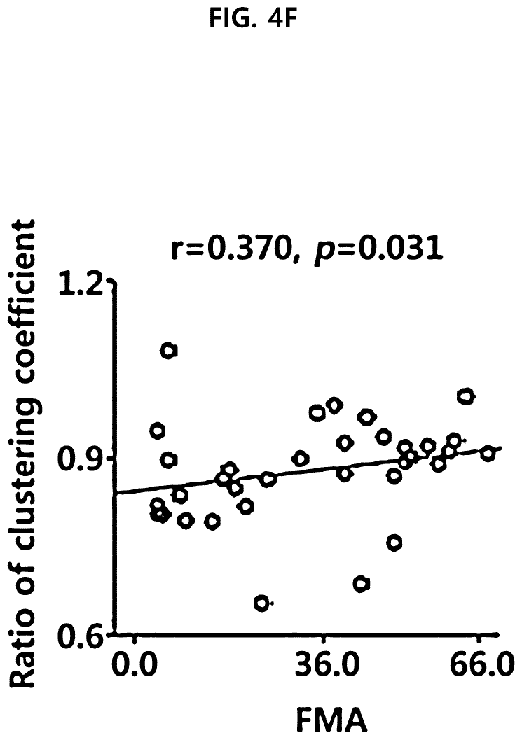

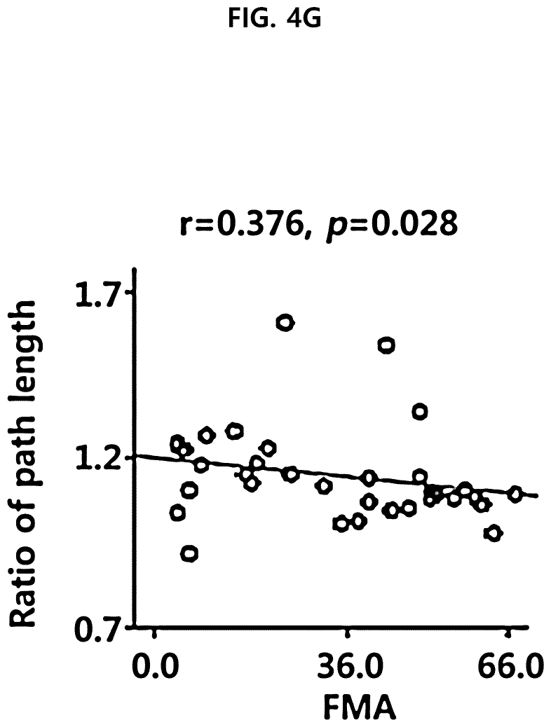

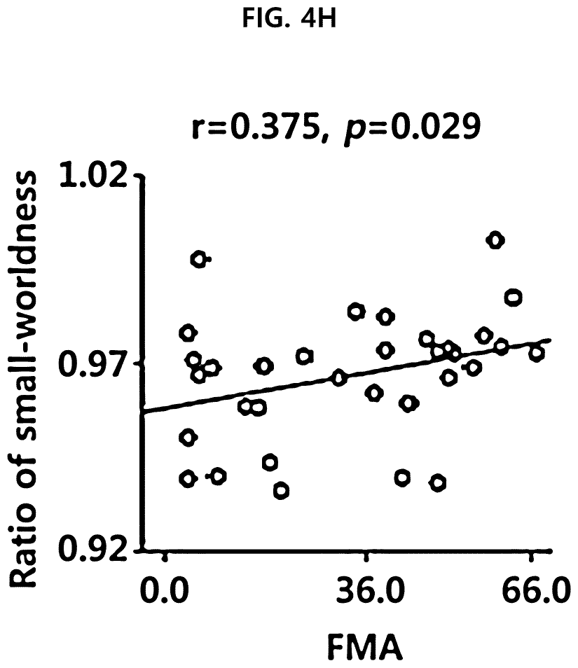

Significant correlation results were found when applying the functional brain network index on the ipsilesional side. The estimated alpha-ipsilesional brain network index during the hand movement task with motor impairment was significantly correlated with the FMA score. Ipsilesional brain network indices of strength (rho=0.340, p=0.049), clustering coefficient (rho=0.342, p=0.048) and small-worldness (rho=0.444, p=0.008) were positively correlated with FMA scores. On the other hand, path length (rho=0.350, p=0.042) was negatively correlated with FMA score, as shown in FIG. 4A to 4D.

Additionally, a significant correlation was observed when using the ratio of alpha brain network indices between the ipsilesional and contralesional hemispheres. The ratios of strength (rho=0.369, p=0.032), clustering coefficient (rho=0.370, p=0.031), and small-worldness (rho=0.375. p=0.029) showed positive correlations with the FMA score, and the ratio of path length (rho=0.376, p=0.028) was negatively correlated with FMA score, as shown in FIG. 4E to 4H.

A significant correlation between the network index and the FMA score of the ipsilesional hemisphere was found in the estimated low-beta frequency band during the hand movement task with motor impairment. Brain network indices on the ipsilesional side of strength (rho=0.328, p=0.058) and clustering coefficient (rho=0.338, p=0.051) were positively correlated with FMA score, while path length (rho=0.340, p=0.049) was negatively correlated with the FMA score.

Overall, according to one embodiment of the present invention. reduced ERD, reduced strength, reduced clustering coefficient. and extended path length were confirmed during movement of the hand without motor impairment compared to movement of the hand with motor impairment. Additionally, a significant correlation was confirmed between the FMA score and the brain network indices. Through this, it was found that the brain network indices identified in one embodiment of the present invention can be a useful brain biomarker for motor impairment, especially post-stroke motor impairment. Therefore, it was confirmed that by using the method according to an embodiment of the present invention, it is possible to effectively provide information on prognosis prediction for the motor impairment and motor impairment-related rehabilitation.

What is claimed is:

1. A method for providing motor impairment-related information, comprising:
(a) a step of measuring a brain network index according to movement performance of an affected side part of a subject: and
(b) a step of comparing the brain network index measured in step (a) with a brain network index according to movement performance of an unaffected side part of the subject.

2. A method for providing motor impairment-related information, comprising:
(a) a step of measuring a brain network index according to movement performance of a subject with motor impairment; and
(b) a step of comparing the brain network index measured in step (a) with a brain network index according to movement performance of a normal subject.

3. The method according to claim 1,
wherein the brain network index comprises one or more selected from the group consisting of strength, clustering coefficient, path length, and small-worldness; or
wherein the brain network index is measured in one or more of an alpha wave region and a low-beta wave region; or
wherein the subject is measured from a subject performing a preset task in step (a); or
wherein the step (a) is performed by electroencephalography (EEG); or
wherein the motor impairment is a stroke; or
wherein the subject in step (a) has upper limb paralysis following a stroke; or
wherein the motor impairment-related information comprises information regarding one or more of prognosis prediction for the motor impairment, motor impairment-related diagnosis, and motor impairment-related rehabilitation.

4. The method according to claim 2,
wherein the brain network index comprises one or more selected from the group consisting of strength, clustering coefficient, path length, and small-worldness; or
wherein the brain network index is measured in one or more of an alpha wave region and a low-beta wave region; or
wherein the subject is measured from a subject performing a preset task in step (a); or
wherein the step (a) is performed by electroencephalography (EEG); or
wherein the motor impairment is a stroke; or
wherein the subject in step (a) has upper limb paralysis following a stroke; or
wherein the motor impairment-related information comprises information regarding one or more of prognosis prediction for the motor impairment, motor impairment-related diagnosis, and motor impairment-related rehabilitation.

5. (canceled)

6. (canceled)

7. (canceled)

8. (canceled)

9. (canceled)

10. A device for providing motor impairment-related information that provides a brain network index obtained according to movement performance of a subject.

11. The device according to claim 10,
wherein the brain network index is expressed as (i) a ratio of a brain network index according to movement performance of the affected side part of the subject to a brain network index according to movement performance of the unaffected side part of the subject, or (ii) a ratio of a brain network index according to movement performance of a subject with motor impairment to a brain network index according to movement performance of a normal subject.

12. A device comprising:
the device for providing motor impairment-related information according to claim 10; and
one or more of a brain stimulation unit that is connected to the device for providing motor impairment-related information and performs brain stimulation based on the brain network index and a rehabilitation assistant unit that assists rehabilitation of motor impairment.

13. The device according to claim 12,
wherein the brain stimulation unit performs one or more of brain electrical stimulation, brain magnetic stimulation, and neurofeedback, and the rehabilitation assistant unit comprises a robot arm that assists rehabilitation.

14. The device according to claim 12,
wherein the device further comprises an instruction device comprising an AR or VR device for instructing a subject movement task.

15. A computer-readable recording medium that is readable by a computer and stores program instructions operable by the computer,
wherein when the program instructions are executed by a processor of the computer, the computer-readable recording medium causes the processor to perform the method for providing motor impairment-related information according to claim 1.

16. A computer-readable recording medium that is readable by a computer and stores program instructions operable by the computer,
wherein when the program instructions are executed by a processor of the computer. the computer-readable recording medium causes the processor to perform the method for providing motor impairment-related information according to claim 2.
